(19) United States
(12) Patent Application Publication
(10) Pub. No.: US 2004/0147433 A1
(43) Pub. Date: Jul. 29, 2004

(54) Neuroimmunophilins for selective neuronal radioprotection

(76) Inventors: Marcus Keep, Honolulu, HI (US); Eskil Elmer, Lund (SE)

Correspondence Address: BIRCH STEWART KOLASCH & BIRCH, PO BOX 747, FALLS CHURCH, VA, 22040-0747, US

(21) Appl. No.: 10/757,533

(22) Filed: Jan. 15, 2004

Related U.S. Application Data
(63) Continuation of application No. 09/787,861, filed on Jun. 14, 2001.
Application No. 09/787,861 is a 371 of international application No. PCT/US98/20040, filed on Sep. 23, 1998.

(51) Int. Cl. A61K 38/13

(52) U.S. Cl. 514/11

(57) ABSTRACT

Method for selectively reducing mammal neuron damage or death in neuroimmunophilin-rich neurons of central, peripheral, and autonomic nervous systems of a mammal while not reducing damage or death to neuroimmunophilin-poor cells and tissues selected from the group consisting of glia, glia-derived tumor cells, abnormal neuron-derived tumor cells, non-brain tumors, and non-neuron tissue of the body from ionizing radiation. The method includes preparing a dosage of a neuroimmunophilin ligand selected from the group consisting of cyclosporins and functional derivatives, metabolites, variants, and salts thereof which are able to cross the blood-brain barrier. The dosage is from an effective amount to less than 1 gr/kg of body weight of said mammal. The method includes the step of administering that dosage to the mammal before, co-incident with, or after ionizing radiation of the mammal. The dose is administered the same day as, but not later than one week after, last radiation exposure.

This application is a continuation of application Ser. No. 09/787,861, which was filed on Jun. 14, 2001. Ser. No. 09/787,861 was a U.S. national phase application of PCT/US98/20040, which was filed on Sep. 23, 1998. Applicants claim the benefits under 35 U.S.C. §120 of the filing dates of both said applications. The entire disclosure of each of said applications is hereby expressly incorporated by reference.

### BACKGROUND OF THE INVENTION

Neuroimmunophilin ligands. Both cyclosporin and FK506 are neuroimmunophilin ligands, that is they bind specifically to neuroimmunophilins. The neuroimmunophilins were previously named after their respective binding ligand i.e. they were defined as cyclophilins and FK-binding proteins. Because the effect of cyclosporin and FK506 on the immune system is so robust and well known in clinical transplantation, the cyclophilin and FK-binding protein families together became known as immunophilins. When it was discovered that neurons were 20 times more enriched in immunophilins than immune cells, the name became neuroimmunophilins. In addition, it was realized that neuroimmunophilin ligands were neuroprotective.

However, it has never been proposed or realized that the differential distribution of neuroimmunophilins could be exploited to improve the safety and efficacy of radiation treatments of the brain, or radiation fields or rays that pass through the brain. The crucial realization is that neurons are highly enriched in neuroimmunophilins and that the glia or support cells of the brain contain little or no neuroimmunophilin protein.

Neuroimmunophilin ligands are herein defined as all compounds that bind to the neuroimmunophilins. Neuroimmunophilin ligands include but are not limited to the immunosuppressants cyclosporin A, cyclosporins, FK506, all their immunosuppressant and non-immunosuppressant analogs, derivatives and variants, as well as small molecule immunophilin ligands developed by the companies Guilford Pharmaceuticals Inc. and Vertex Pharmaceuticals Inc. and described in other patent applications. Treatment medication or treatment medications will be defined as a medicament comprising as its active ingredients not less than one neuroimmunophilin ligand, and may contain a mixture of two or more similar or different neumimmunophilin ligands. The three main classes of neuroimmunophilin ligands are discussed below, including cyciosporins, FK506 and the small FK-binding protein neuroimmunophilins ligands (“FKBP-neuroimmunophilin ligands”) of Guilford Pharmaceuticals Inc. and Vertex Pharmaceuticals Inc.

Cyclosporin A and derivatives. It is already known that cyclosporin A is an immunosuppressive drug. The above mentioned treatment medication has already been described, in United States Pat. No. 4,117,118 and numerous patents since, which relate to its production, formulation and immunosuppressive properties.

Cyclosporin A is a product of the fungus Tolypocladium Inflatum Gams. It is a cyclic poly-amino acid molecule, consisting of 11 amino acids. One of the amino acids is unique for cyclosporin A, a β-hydroxyamino acid called butenyl-methyl-threonin (MeBmt). The molecular weight is 1202.6 and the chemical composition is C₆₂H₁₁₁N₁₁0₁₂.

The molecule is highly lipophilic, and therefore virtually insoluble in water. The bioavailability after an oral dose varies between 8 and 60% depending in part on the bile flow. The drug is absorbed mainly in the small intestine. The drug is transported in the blood within red blood cells to about 58%, and the remaining approximately 10-20% in leukocytes, and 33% bound to plasma proteins. In the plasma cyclosporin A is bound to high-density lipoprotein, low-density lipoproteins, very-low density lipoproteins and a small fraction to albumin. A very small fraction is free in plasma.

The drug undergoes extensive metabolism, mainly in the liver by the cytochrome P450 system. There are at least 30 known metabolites of cyclosporin A, with various chemical modifications, such as hydroxylation, demethylation, oxidation and epoxide formations. There are a number of variants of cyclosporin A, differing for example in one amino acid, which have similar pharmacological properties. Under normal conditions, cyclosporin A and its metabolites do not pass the blood-brain barrier. When the glycoprotein-p transporter is poisoned, or the blood-brain barrier is disrupted, cyclosporin is able to cross it and come into contact with neurons. Several analogs of cyclosporin are able to readily cross the blood-brain barrier. Several analogs of cyclosporin are not immunosuppressants. There is a subset of analogs of cyclosporin that both readily cross the blood-brain barrier and are not immunosuppressants.

This entire family of cyclosporins, all derivatives, variants, amino acid variants, metabolites, including variations of mono-, di- and trihydroxylates, N-demethylates, aldehydes, carboxylates, conjugates, sulphates, glucuronides, intramolecular cyclizations and those without a cyclic structure as well as shorter peptides and amino acids and their derivatives and salts with or without immunosuppressive properties and whether able to cross the blood brain barrier or not will hereinafter be referred to as cyclosporins. Cyclosporins will hereinafter be referred to as “neuroimmunophilin ligand or ligands” based on their affinity and binding to the group of neuroimmunophilins called cyclophilins.

The present invention also discloses treatment medications of the family of cyclosporins and all known salts, variants, amino acid variants, derivatives, metabolites and their salts and derivatives for use in treatments of the conditions listed below, as well as the use of such treatment medications for the treatment of such conditions. This includes cyclosporin A, cyclosporin C, cyclosporin D, cyclosporin G. In addition, this includes all products of the fungus Tolypocladium Inflarum Gams. Some known metabolites of cyclosporin A include the following: (according to Hawk 's Cay nomenclature) AM1, AM9, AM1c, AM4N, AM19, AM1c9, AM1c4N9, AM1A, AM1Ac, AM1A4N, AM1Ac, AM1AL, AM11d, AM69, AM4N9, AM14N, AM14N9, AM4N69, AM99N, Dihydro-CsA, Dihydro-CsC, Dihydro-CsD, Dihydro-CsG, M17, AM1c-GLC, sulphate conjugate of cyclosporin, BHlla, BH15a, B, G, E, (and with come overlap with the Hawk's above, according to Maurer 's nomenclature) M1, M2, M3, M4, M5, M6, M7, M8, M9, M10, M11, M12, M13, M14, M15, M16, M17, M18, M19, M20, M21, M22, M23, M24, M25, M26, MUNDFI and MeBMT. Some metabolites of cyclosporin G include GM1, GM9, GM4N, GM1c, GM1c9, and GM19. Modified cyclosporins include modified C-9 amino acid analogs, modified 8-amino acid analogs, modified 6-position analogs containing MeAla or MeAbu residue, and SDZ 209-313, SDZ-205-549, SDZ-033-243, SDZ IMM 125 and SDZ-PSC-833.

FK506 and its derivatives. FK506 is a macrolide compound, known and disclosed in European Patent Publication No. 0184162 and other documents. The known macrolide compounds include FR-900506, FR-900520, FR-900523 and FR-900525 isolated from microorganisms of the genus Streptomyces like Streptomyces tsukubensis No. 9993 and their related compounds. Derivatives include ascomycin (C21-ethyl-FK506), C18-OH-ascomycin, 9-deoxo-31-0-demethylFK506, 31-0-demethylFK506, C32-indolyl-ascomycin, A-119435, L-683,590, L-685,818 and L-688,617. These compounds were indicated as useful in treating rejection in transplantation, autoimmune diseases, and in U.S. Pat. No. 5,642,351 as useful for preventing or treating cerebral ischemic disease. FK506 and its derivative macrolide compounds and salts with or without immunosuppressive properties will hereinafter be referred to as FKs. FKs will hereinafter be referred to as a “neuroimmunophilin ligand or ligands” based on their affinity and binding to the group of neuroimmunophilins called FK-binding proteins, especially FKBP12, or other FKBPs.

Guilford and Vertex have discovered a series of small molecules which easily enter the brain and have been found to be neurotrophic and neuroprotective, by virtue of their ability to bind as ligands to FKBP12 and FKBPs, for which they hold a variety of patents including U.S. Pat. Nos. 5,780,484 and 5,614,547. However they do not claim protection from ionizing radiation damage. Further they do not claim that using these small molecule FKBP-type neuroimmunophilin ligands would be an improvement over current techniques of ionizing radiation treatment, or protection from ionizing radiation exposure. Small molecule FKBF-type neuroimmunophilin ligands will hereinafter be refereed to as a “neuroimmunophilin ligand or ligands” based on their affinity and binding to the group of neuroimmunophilins called FK-binding proteins, especially FKBP12, or other FKBPs.

Currently under development are small molecules which easily enter the brain which have neurotrophic and neuroprotective properties by virtue of their ability to bind to the neuroimmunophilin cyclophilin. It has not been claimed that using these small molecule cyclophilin-type neuroimmunophilin ligands would be an improvement over current techniques of ionizing radiation treatment, or from ionizing radiation. Cyclophilin-type neuroimmunophilin ligands will hereinafter be referred to as “neuroimmunophilin ligand or ligands” based on their affinity and binding to the group of neuroimmunophilins called cyclophilins.

A dose of ionizing radiation causes damage and kills cells primarily by ionizing water or oxygen into toxic hydroxyl, oxygen and/or other species of free radicals. These radicals then damage or kill the cell by their high reactivity against cell proteins, membranes and DNA. In addition, the free radicals themselves can induce a mitochondrial permeability transition which incapacitates a cells ability to make ATP to carry out its normal functions and causes the mitochondria to release mitochondtial enzymes which activate nuclear caspases and other enzymes that cause apoptosis, or programmed cell death.

Cyclosporins, but not FK506, nor the FKBP-type neuroimmunophilin ligands, blocks the formation of this mitochondrial transition and thereby blocks apoptosis. This will make cyclosporins most likely the most effective of the neuroimmunophilin ligands, though a mixture with one or more other ligands may have a synergistic effect.

Radiation therapy. Below is a description of the art of radiation treatment for cancer and other conditions. Never before has it been suggested that radiation therapy could be improved by the use of a selective neuron-protecting drug. Never before has it been proposed that by administering a drug of the class of neuroimmunophilin ligands that it would selectively improve the resistance of normal neurons which are neuroimmunophilin-rich in brain, spinal cord and peripheral nerves to the toxic effects of ionizing radiation, compared to all other types of cells which are neuroimmunophilin-poor. Never before has it been realized that most primary brain cancers arise from neuroimmunophilin-poor glial cells (gliomas) or astrocytes (astrocytomas) or oligodendrocytes (oligodendrogliomas), and thus would not be protected from the toxic effects of ionizing radiation, while normal neuroimmunophilin-rich neurons would be protected from ionizing radiation by a neuroimmunophilin ligand. Thus the person that is systemically treated with a radioprotecting neuroimmunophilin ligand would have selective and improved protection of neurons, improving the art of radiation treatment in a non-obvious and novel way.

Ionizing radiation is frequently used in the medical field to treat disease. Primary brain tumors are often treated with radiotherapy, and are radiated with a wide field including much or all of the brain with an X-ray source such as a linear accellerator over one or many daily sessions typically over eight weeks. Sometimes the radiation is from gamma rays or proton and particle beam. This radiation slows the growth of the brain tumor, but also kills normal neurons. Cystic brain tumors sometimes have radioactive liquids instilled into them. Sometimes radioactive pellets are temporarily or permanently implanted.

Metastatic tumors from lung, breast, colon, skin and other organs often go to the brain. There are tumors of the head that are adjacent to brain, such as pituitary tumors, meningiomas and craniopharyngiomas. There are radiosensitive vascular malformations in the brain. There are disorders of the brain which can be helped with partial or complete lesions of small brain structures including Parkinson's disease, epilepsy, obsessive compulsive disorder and trigeminal neuralgia, in which radiation passes through normal brain. These tumors and conditions are often treated either with radiotherapy as described above or radiosurgery. Radiosurgery uses either gamma rays or X-rays usually administering a high dose precisely localized in one session, with radiation passing through normal brain enroute and beyond the target structure.

Tumors in the body, such as squamous cell, laryngeal, lung, breast, renal, or prostate cancers are often treated with radiation by linear accellerator, or implantation of radioactive pellets. The radiation fields treating these cancers sometimes include neural structures of the brain, spinal cord or peripheral nerves.

In addition to therapeutic medical uses for radiation, there are non-medical instances of radiation exposure. They include the accidental dosage or overdosage by radioactive substances, and supratherapeutic dosage using a medical radiation device. Occasionally there is the inadvertent expose of a pregnant person's fetus, and thus its developing nervous system, to X-ray radiation.

Occupational or accidental situations of radiation exposure such as nuclear reactor radiation leak, cause radiation of the brain in addition to the rest of the body.

### SUMMARY OF THE INVENTION

The Instant Invention. There are side effects of radiation. It causes normal neurons to die, causing nausea and vomiting, lethargy, permanent decreased cognition, drop in intelligence, lost endocrine control, radiation necrosis and loss of function, spinal cord dysfunction and necrosis with resultant paralysis. The concern about these resulting side effects reduces the radiation doses that can be given by radiation oncologists, producing fewer cures, or faster recurrence than would be possible if higher doses could be given. In addition, the pediatric population is more susceptible to radiation effects of the nervous system, causing mental retardation. If neurons could be protected, these side effects could be decreased or prevented leading to more cancers cured or more effectively treated cancers.

There is a need for a treatment that protects normal neurons from radiation, while leaving tumor cells susceptible. Treating a person exposed to radiation with neuroimmunophilin ligands would be a significant improvement over current radiation treatment. Being able to administer such a compound to patients has industrial applicability,

The simultaneous realization of three factors leads to the non-obvious and novel inventive step that giving neuroimmunophilin ligands to radiation therapy patients would selectively protect normal neurons over tumor cells and especially brain tumor cells, and thus improve radiation therapy—(1) that neurons are more enriched in neuroimmunophilins than any other tissue (especially compared to brain cancer or other cancer cells), (2) that drugs of the class of neuroimmunophilin ligands, notably cyclosporin and FK506, are protective to cells containing neuroimmunophilins from free radicals, and (3) that ionizing radiation kills cells via the production of free radicals. This also leads to the non-obvious inventive step that persons exposed to non-medical toxic doses of whole body radiation might better survive, or survive longer if their neurons were selectively protected compared to not being protected at all.

### DETAILED DESCRIPTION OF THE INVENTION

Medicament and administration. Administration of the treatment medication may be by any suitable route including oral, sublingual, buccal, nasal, inhalation, parenteral (including intraperitoneal, intraorgan, subcutaneous, intradermal, intramuscular, intra-articular, venous (central, hepatic or peripheral), lymphatic, cardiac, arterial, including selective or superselective cerebral arterial approach, retrograde perfusion through cerebral venous system, via catheter into the brain parenchyma or ventricles), direct exposure or under pressure onto or through the brain or spinal tissue, or any of the cerebrospinal fluid ventricles, injections into the subarachnoid, brain cisternal, subdural or epidural spaces, via brain cisterns or lumbar puncture, intra and peri-ocular instillation including application by injection around the eye, within the eyeball, its structures and layers, as well as via enteral, bowel, rectal, vaginal, urethral or bladder cistemal. Also for in utero and perinatal indications then injections into the maternal vasculature, or through or into maternal organs, and into embryo, fetus, neonate and allied tissues and spaces such as the amniotic sac, the umbilical cord, the umbilical artery or veins and the placenta, with parenteral being the preferred route. The preferred route may vary depending on the condition of the patient.

Included in the invention is administration of the treatment medication via any means with purposeful disruption of brain or spinal parenchyma, or disrupting the blood-brain barrier via mechanical, thermal, cryogenic, chemical, toxic, receptor inhibitor or augmentor, p-glycoprotein transporter poisoning, inhibition or saturation, osmotic, charge altering, radiation, photon, electrical or other energy or process.

This invention includes all methods of administering treatment medications along with all methods of opening, bypassing or disrupting the blood-brain barrier in combination, simultaneously or in sequence to get the treatment medication in contact with nervous tissues in order for it to exert neuro-radioprotection.

This invention includes the possibility of the timing and sequence of delivery of treatment medications to include pre-treatment, as well as simultaneous with treatment.

While it is possible for the treatment medication to be administered alone, it is preferred to present it as part of a pharmaceutical formulary drug. The formulary drug of this invention comprise at least one administered treatment medication as defined above together with one or several appropriate carriers thereof and possibly other pharmaceutical treatment medications. The carriers must be appropriate in that they can readily coexist with the other agents of the formulary drug and are not detrimental to the receiver thereof. This treatment medication combined, as described in this paragraph, with other appropriate agents common to the art, is defined herein as the formulary drug.

The formulary drug includes those suitable for administration by the routes including oral, sublingual, buccal, nasal, inhalation, parenteral (including intraperitoneal, intraorgan, subcutaneous, intradermal, intramuscular, intra-articular, venous (central, hepatic or peripheral), lymphatic, cardiac, arterial, including selective or superselective cerebral arterial approach, retrograde perfusion through cerebral venous system, via catheter into the brain parenchyma or ventricles), direct exposure or under pressure onto or through the brain or spinal tissue, or any of the cerebrospinal fluid ventricles, injections into the subarachnoid, brain cistemal, subdural or epidural spaces, via brain cisterns or lumbar puncture, intra and peri-ocular instillation including application by injection around the eye, within the eyeball, its structures and layers, as well as via enteral, bowel, rectal, vaginal, urethral or bladder cisternal. Also for in utero and perinatal indications then injections into the maternal vasculature, or through or into maternal organs including the uterus, cervix and vagina, and into embryo, fetus, neonate and allied tissues and spaces such as the amniotic sac, the umbilical cord, the umbilical artery or veins and the placenta, with parenteral being the preferred route.

The formulary drug may be distributed and made available in convenient unit dose form such as capsules and ampoules, containing the treatment medication of the invention, and may be manufactured and distributed by any of the methods known to the pharmaceutical arts. In addition to the treatment medication, the formulary drug can also contain other usual agents of the art relating to the type of formulary drug produced. The formulary drug may, by example, take the configuration of suspensions, solutions and emulsions of the treatment medication in lipid, non-aqueous or aqueous dilutents, solvents, dissolving agents, emulsifiers, syrups, granulates or powders, or mixtures of these. The formulary drug can also contain coloring agents, preservatives, perfumes, flavoring additions and sweetening agents. In addition to the treatment medication, the formulary drug can also contain other pharmaceutically active medications. The manufacture and distribution of the formulary drug is carried out by techniques known to the art, such as, evenly and intimately bringing together the treatment medication with liquids or fine solids or both, and then if needed, forming the formulary drug into a dose unit form. The discrete dose, portion and carrier vehicle constituting the formulary drug will generally be adapted by virtue of shape or packaging for medical administration and distributed for this purpose.

The formulary drug acceptable for oral administration may be manufactured and distributed as individual dosage units such as capsules, pills, tablets, dragees, dissolvable powders, or cachets, each containing a known dose of the treatment medication; as powder or granules; as solution or suspension in syrups, elixirs as a lipid, aqueous liquid or a non-aqueous liquid; or as an oil-in-water emulsion or as a water-in-oil emulsion.

Tablets can be manufactured and distributed by compression or mould, from treatment medication possibly with one or more additional pharmaceutically active compound. Compressed tablets can be manufactured and distributed through compression in a machine typical to the art a known quantity of the treatment medication in a dispersible configuration such as powder or granules, possibly mixed with other agents including binders, lubricants, inert dilutents, preservatives, and dispersing agents. Moulded tablets can be manufactured and distributed by moulding in a machine typical to the art a mix of known quantity of treatment medication addition pharmaceutically active compounds and other additives moistened with a liquid dilutent. The tablets can possibly be coated, enveloped or covered, with substances including protective matrices, which can contain opacifiers or sweeteners and can be formulated to allow slow or controlled release, or also release within a certain part of the digestive system of the contained treatment medications. Capsules can be manufactured and distributed by placement of a known quantity of treatment medication, additional pharmaceutically active compounds and additives within a two part or sealed capsule of gelatin or other aqueous dissolvable substance. The treatment medication can also be manufactured and distributed as formulary drug in microencapsulated, microsomal, micellar and microemulsion forms.

The formulary drug containing the treatment medication acceptable for parenteral administration can be manufactured and distributed from aqueous and non-aqueous sterile injection solutions, other pharmaceutically active compounds, additives including antioxidants, bacteriostats and solutes and sugars such as mannitol to make the formulary drug isotonic, hypotonic or hypertonic with the blood of the recipient; and also aqueous and non-aqueous sterile suspensions which can include suspenders and thickeners. The formulary drug can be manufactured and distributed in unit-dose or multi-dose containers, such as sealed glass or plastic ampoules, vials, bottles and bags as a liquid, and in a dry state requiring only the addition of sterile liquid, for example water, saline or dextrose solutions, immediately prior to use. Extemporaneous solutions and suspensions for injection can be prepared from powders and tablets of the kind above described.

The formulary drug-containing the treatment medication acceptable for administration into the brain and related structures, spinal cord and related structures, ventricular system and cerebrospinal fluid spaces can be manufactured and distributed from aqueous and non-aqueous sterile injection solutions, other pharmaceutically active compounds, additives including anti-oxidants, bacteriostats and solutes and sugars such as mannitol to make the formulary drug isotonic, hypotonic or hypertonic with the cerebrospinal fluid; and also aqueous and non-aqueous sterile suspensions including solvents which can include suspenders and thickeners. The formulary drug can be manufactured and distributed in unit-dose or multi-dose containers, such as sealed glass or plastic ampoules, vials, bottles and bags as a liquid, and in a dry state requiring only the addition of sterile liquid, for example water, saline or dextrose solutions, immediately prior to use. Extemporaneous solutions and suspensions for injection can be prepared from powders and tablets of the kind above described.

The desired unit dose of formulary drug are those containing a daily dose or ionizing radiation treatment dose or an appropriate fraction thereof, of the administered treatment medication. Unit dose forms of the invention may also include more complex systems such as double barrelled syringes, syringes with sequential compartments one of which may contain the treatment medication, and the other any necessary dilutents or vehicles, or agents for opening the blood-brain barrier. The agents in the syringes would be released sequentially or as a mixture or combination of the two after the triggering of the syringe plunger. Such systems are known in the art.

The formulary drug generally contains from 0.1 to 90% of the treatment medication by weight of the total composition. Amounts of from 0.0001 mg to 200 mg/kg, or preferably 0.001 to 50 mg/kg, of body weight per day for parenteral administration and 0.001 to 150 mg/kg, preferably 0.01 to 60 mg/kg, of body weight per day for enteral administration, can be given to improve neuro-radioprotection. Nevertheless, it could be necessary to alter those dosage rates, depending on the condition, weight, and individual reaction of the subject to the treatment, the type of formulary drug in which the treatment medication is administered and the mode in which the administration is carried out, and the stage of the disease process or interval of administration. It may thus be sometimes adequate to use less than the before stated minimum dose, while in other instances the upper limit must be surpassed to obtain therapeutic results.

The invention is for the use of the treatment medication in the conditions described throughout the application. The invention thus also includes all advertising, labelling, packaging, informational materials, inserts, product descriptions, advertising materials, the written word, including letter, pamphlet, brochures, magazines and books, as well as other media of communication including the spoken word, fax, phone, photos, radio, video, television, film, internet, e-mail or computer based, and proposals for clinical trials and study protocols for clinical trials using the treatment medication for its selective neuronal protection from ionizing radiation.

### EXAMPLES

Examples 1-14 demonstrate typical situations where neuro-radioprotection in accordance with this invention can be used. Examples 15-27 demonstrate typical neuroimmunophilin ligand formulations for administration as neuro-radioprotective drugs.

### Example 1

A patient has a primary brain tumor, such as an astrocytoma, oligodendroglioma or ependymoma and is a candidate for clinical radiation therapy, radiosurgery or brachytherapy. Four hours before radiation treatment, the patient has an injection of a neuroimmunophilin ligand into the vein, artery, thecal sac (via lumbar puncture) or ventricular catheter. The patient then has a session of clinical radiation treatment. Because the neuroimmunophilins are concentrated in neurons but not glial tumors, the drug is concentrated in the neurons but not the tumor. Fewer neurons die compared to tumor at a given radiation dose compared to untreated patients, increasing the safety of higher radiation doses to kill tumor, and reducing the loss of neurons.

### Example 2

A patient with a primary brain tumor such as an astrocytoma, anaplastic astrocytoma or glioblastoma multiforme receives X-radiation therapy to the brain for a series of daily treatments over two months. This radiation field is wide and include large areas of normal brain in addition to the normal neurons adjacent to tumor. During the period of radiation therapy, to protect the brain neurons from radiation damage, or allow the administration of larger doses of radiation than otherwise tolerated, the patient is given a series of doses of neuroimmunophilin ligand. This reduces side effects of cognitive decline, brain swelling, nausea, headaches and radiation necrosis. This increases the chances for cure or control of tumor growth.

### Example 3

A patient with a pituitary tumor is going to have radiation therapy or radiosurgery. Part of the radiation field includes the optic chiasm, optic nerve and optic tract. To protect the optic chiasm, nerve and tract neurons from radiation damage, and the patient from vision loss, or blindness, the patient is given a dose of neuroimmunophilin ligand prior to each session.

### Example 4

A patient with a craniopharyngioma is going to have radiation therapy or radiosurgery. Part of the radiation field includes the hypothalamus of the brain. To protect the hypothalamic neurons from radiation damage, the patient is given a dose of neuroimmunophilin ligand, prior to each session. This reduces the side effects of endocrine abnormalities or insufficiencies, diabetes insipidus, retardation or mental decline and radiation necrosis.

### Example 5

An infant or child with a medulloblastoma brain tumor requires whole brain radiation, including the forebrain, midbrain, cerebellum, brain stem and spinal cord. To protect all the neurons in these locations, the infant or child is given a dose of neuroimmunophilin ligand prior to each session. This reduces the common side effects of mental retardation, cognitive and functional decline, endocrine abnormalities and radiation necrosis. This allows the treatment to be given at an earlier age than without neuroradioprotection. This allows a higher radiation dose be given than would be allowed without neuroradioprotection.

### Example 6

A patient with one or more metastatic tumors from a lung, breast or other primary cancer to the brain has Gamma Knife, particle beam or Linear accellerator based stereotactic radiosurgery, with the gamma, particle beam or X-radiation fields including normal brain neurons. To protect the normal brain neurons in the path of the radiation, the patient is given a dose of neuroimmunophilin ligand. This reduces the side effects of radiation necrosis and cognitive decline.

### Example 7

A patient with a lung tumor is going to have lung radiation therapy. Part of the radiation field includes the spinal cord. To protect the spinal cord neurons from “bystander” radiation damage, the patient is given a dose of neuroimmunophilin ligand prior to each session.

### Example 8

A patient with a kidney cancer is going to have kidney radiation therapy. Part of the radiation field includes the small and large bowel. To protect the autonomic neurons in the bowel from “bystander” radiation damage, the patient is given a dose of neuroimmunophilin ligand prior to each session.

### Example 9

A patient with prostate cancer is going to have radiation therapy or brachytherapy radioactive prostate implants. Part of the radiation field includes the pudendal nerves controlling penile sensation, erection and ejaculation. To protect the penile nerves passing adjacent to the prostate from “bystander” radiation damage, the patient is given a dose or doses of neuroimmunophilin ligand. This reduces impotence.

### Example 10

A patient with a breast tumor is going to have radiation therapy. Part of the radiation field includes the brachial plexus nerves. To protect the brachial plexus nerves that innervate the muscles and skin of the arm from “bystander” radiation damage, the patient is given a dose of neuroimmunophilin ligand prior to each session. This reduces the side effect of loss of sensorimotor function to the arm.

### Example 11

Staff of a uranium processing plant is exposed to radiation. In order to protect the neurons of the people exposed, they are administered an intravenous dose of cyclosporin A and/or FK506. This reduces radiation poisoning and increases chances for survival.

### Example 12

A person is in an occupation or situation with high likelihood of radiation exposure, or has just received whole body radiation. The person is administered or self-administers a dose of neuroimmunophilin ligand to protect all the neurons in his or her body and increases chances for survival.

### Example 13

A person is in earth orbit or space travel and receives cosmic radiation. The person is administered dose or doses of neuroimmunophilin ligand to protect all neurons in his or her body and increase chances for survival.

### Example 14

A person is pregnant and the fetus is exposed to radiation. To reduce the damage to developing fetal neurons and brain, and reduce brain damage and mental retardation of the surviving child, a dose of neuroimmunophilin ligand is administered.

### Example 15

### Sterile Injectable Concentrate Formulary Drug

Containing per ml:

| | |
|---|---|
| Cyclosporin A | 100 mg |
| Spiritus fortis | 415 mg |
| Polyoxyethylated castor oil | 600 mg |

The formulary drug is sterilized by heat or radiation and then placed in a sealed container such as glass in doses of 1 or 5 ml. The sterile injectable concentrate formulary drug is diluted in 20 ml saline so that it may be administered by infusion or by injection into artery, vein, brain, spine or cerebrospinal fluid spaces.

### Example 16

### Sterile Injectable Concentrate Formulary Drug

Containing per ml:

| | |
|---|---|
| Cyclosporin A | 200 mg |
| Tween 80 | 800 mg |

The formulary drug is sterilized by heat or radiation and then placed in a sealed container such as glass in doses of 1 or 5 ml. The sterile injectable concentrate formulary drug is diluted 1 ml in 10 ml saline so that it may be administered by infusion or by injection into artery, vein, brain, spine or cerebrospinal fluid spaces.

### Example 17

### Capsule Formulary Drug

| | |
|---|---|
| Cyclosporin A | 200 mg |
| Iron oxide E172 | 1 mg |
| Titanium dioxide | 3 mg |
| Ethanol | 100 mg |
| Corn oil | 415 mg |
| Gelatine | 280 mg |
| Labrafil | 300 mg |
| Andrisorb | 105 mg |
| Glycerol 85% | 3 mg |

A one or two part capsule is prepared by placing the formulary drug in a one or two part gelatine capsule.

### Example 18

### Liquid Oral Formulary Drug

Containing per 1 ml:

| | |
|---|---|
| Cyclosporin A | 200 mg |
| Ethanol | 100 mg |
| Corn oil | 430 mg |
| Labrafil | 200 mg |

### Example 19

### Sterile Injectable Concentrate Formulary Drug

Containing per ml

| | |
|---|---|
| FK506 anhydrous | 5 mg |
| Polyoxyl 60 hydrogenated castor oil | 200 mg |
| Dehydrated alcohol USP, 80% | v/v |

The formulary drug is sterilized by heat or radiation and then placed in a sealed container such as glass in doses of 1 or 5 ml. The sterile injectable concentrate formulary drug is diluted 1 ml in 10 ml saline so that it may be administered by infusion or by injection into artery, vein, brain, spine or cerebrospinal fluid spaces.

### Example 20

### Capsule Formulary Drug

| | |
|---|---|
| FK506 anhydrous | 5 mg |
| Lactose | 100 mg |
| Hydroxypropyl methylcellulose | 100 mg |
| Croscarmellose sodium | 10 mg |
| Magnesium stearate | 10 mg |

A one or two part capsule is prepared by placing the formulary drug in a one or two part gelatin capsule.

### Example 21

### Sterile Injectable Concentrate Formulary Drug

Containing per ml

| | |
|---|---|
| Small molecule FKBP-type neuroimmunophilin ligand | 5 mg |
| Polyoxyl 6O hydrogenated castor oil | 200 mg |
| Dehydrated alcohol USP, 80% | v/v |

The formulary drug is sterilized by heat or radiation and then placed in a sealed container such as glass in doses of 1 or 5 ml. The sterile injectable concentrate formulary drug is diluted 1 ml in 10 ml saline so that it may be administered by infusion or by injection into artery, vein, brain, spine or cerebrospinal fluid spaces.

### Example 22

### Capsule Formulary Drug

| | |
|---|---|
| Small molecule FKBP-type neuroimmunophilin ligand | 5 mg |
| Lactose | 100 mg |
| Hydroxypropyl methylcellulose | 100 mg |
| Croscarmellose sodium | 10 mg |
| Magnesium stearate | 10 mg |

A one or two part capsule is prepared by placing the formulary drug in a one or two part gelatine capsule.

### Example 23

### Sterile Injectable Concentrate Formulary Drug

Containing per ml:

| | |
|---|---|
| Cyclosporin A | 200 mg |
| FK506 anhydrous | 5 mg |
| Small molecule FKBP-type neuroimmunophilin ligand | 5 mg |
| Tween 80 | v/v |

The formulary drug is sterilized by heat or radiation and then placed in a sealed container such as glass in doses of 1 or 5 ml. The sterile injectable concentrate formulary drug is diluted 1 ml in 10 ml saline so that it, may be administered by infusion or by injection into artery, vein, brain, spine or cerebrospinal fluid spaces.

### Example 24

### Sterile Injectable Concentrate Formulary Drug

Containing per ml:

| | |
|---|---|
| Small molecule cyclophilin-type neuroimmunophilin ligand | 5 mg |
| Polyoxyl 60 hydrogenated castor oil | 200 mg |
| Dehydrated alcohol USP, 80% | v/v |

The formulary drug is sterilized by heat or radiation & then placed in a sealed container such as glass in doses of 1 or 5 ml. The sterile injectable concentrate formulary drug is diluted in 10 ml saline so that it may be administered by infusion or by injection into artery, vein, brain, spine or cerebrospinal fluid spaces.

### Example 25

### Capsule Formulary Drug

| | |
|---|---|
| Small molecule cyclophilin-type neuroimmunophilin ligand | 5 mg |
| Lactose | 100 mg |
| Hydroxypropyl methylcellulose | 100 mg |
| Croscarmellose sodium | 10 mg |
| Magnesium stearate | 10 mg |

A one or two part capsule is prepared by placing the formulary drug in a one or two part gelatine capsule.

### Example 26

### Sterile Injectable Concentrate Formulary Drug

Containing per ml:

| | |
|---|---|
| Cyclosporin A | 200 mg |
| FK506 anhydrous | 5 mg |
| Small molecule FKBP-type neuroimmunophilin ligand | 5 mg |
| Small molecule cyclophilin-type neumimmunophilin ligand | 5 mg |
| Tween 80 | v/v |

The formulary drug is sterilized by heat or radiation & then placed in a sealed container such as glass in doses of 1 or 5 ml. The sterile injectable concentrate formulary drug is diluted 1 ml in 10 ml saline so that it may be administered by infusion or by injection into artery, vein, brain, spine or cerebrospinal fluid spaces.

### Example 27

### Capsule Formulary Drug

| | |
|---|---|
| Cyclosporin A | 200 mg |
| FK506 anhydrous | 5 mg |
| Small molecule FKBP-type neuroimmunophilin ligand | 5 mg |
| Small molecule cyclophilin-type neuroimmunophilin ligand | 5 mg |
| Iron oxide E172 | 1 mg |
| Titanium dioxide | 3 mg |
| Ethanol | 100 mg |
| Corn oil | 415 mg |
| Gelatine | 280 mg |
| Labrafil | 300 mg |
| Andrisorb | 105 mg |
| Glycerol 85% | 3 mg |

A one or two part capsule is prepared by placing the formulary drug in a one or two part gelatine capsule.

What is claimed is:

1. A method for improved radiation treatment by selectively reducing mammal neuron death from ionizing radiation in cyclophilin-rich neurons of central, peripheral, and autonomic nervous systems of a mammal while not reducing damage or death to cyclophilin-poor cells and tissues selected from the group consisting of brain tumors, meningiomas, pituitary tumors, craniopharyngioma, lung tumors, renal tumors, breast tumors, colon tumors, skin tumors, squamous cell tumors, laryngeal tumors, and prostate tumors, said method comprising the steps of:
(a) preparing a dosage of cyclophilin ligand for parenteral or enteral administration, said cyclophilin ligand being selected from the group consisting of cyclosporins and functional derivatives, metabolites, variants, and salts thereof selected from the group consisting of cyclosporin A, cyclosporin C, cyclosporin D, cyclosporin G, cyclosporin AM1, cyclosporin AM9, cyclosporin AM1c, cyclosporin AM4N, cyclosporin AM19, cyclosporin AM1c9, cyclosporin AM1A, cyclosporin AM1A4N, cyclosporin AM1Ac, cyclosporin AM1AL, cyclosporin AM11d, cyclosporin AM69, cyclosporin AM4N9, cyclosporin AM14N, cyclosporin AM14N9, cyclosporin 4N69, cyclosporin AM99N, dihydrocyclosporin CsA, dihydrocyclosporin CsC, dihydrocyclosporin CsD, dihydrocyclosporin CsG, cyclosporin M17, cyclosporin AM1c-GLC, cyclosporin sulfate conjugate, cyclosporin BH11a, cyclosporin BH15a, cyclosporin B, cyclosporin G, cyclosporin E, cyclosporin M1 through cyclosporin M26, cyclosporin MUNDFI, cyclosporin MeBMT, cyclosporin GM1, cyclosporin GM9, cyclosporin GM4N, cyclosporin GM1c, cyclosporin GM1c9, cyclosporin GM19, cyclosporin SDZ-209-313, cyclosporin SDZ-205-549, cyclosporin SDZ-033-243, cyclosporin SDZ-IMM-125, and cyclosporin SDZ-PSC-833, which are able to cross the blood-brain barrier, said dosage being from 0.001 to 50 mg/kg of body weight of said mammal for parenteral administration and from 0.01 to 60 mg/kg of body weight of said mammal for enteral administration; and
(b) administering said dosage to said mammal before administering ionizing radiation treatment to said mammal.

2. An improved method in accordance with claim 1, for ionizing radiation treatment of a patient with a disease of a condition requiring ionizing radiation treatment, employing a selective neuronal ionizing protector, said method comprising:
treating said patient with an effective amount of cyclophilin ligand as the selective neuronal ionizing radiation protector, said cyclophilin ligand being selected from the group consisting of cyclosporins and functional derivatives, metabolites, variants, and salts thereof, which are able to cross the blood-brain barrier.

3. The method of claim 1, wherein said ionizing radiation comprises a radiation which is selected from the group consisting of alpha radiation, beta radiation, X radiation, gamma radiation, cosmic radiation, fast neutron radiation, proton radiation, and particle beam radiation.

4. The method of claim 1, wherein said ionizing radiation exposure is therapeutic treatment radiation from medical sources, or non-therapeutic radiation from industrial sources, natural sources, man-made sources, or nuclear sources.

5. The method of claim 1, wherein said cyclophilin ligand is administered by parenteral injection, said injection being into, or adjacent to, the brain, tumor, or spinal cord, or via cerebrospinal fluid spaces, intraventricular fluid spaces, or intrathecal spaces, or via application into the digestive, respiratory, or genito-urinary systems, or skin, or by a combination of these routes, so that said cyclophilin ligand comes into contact with neurons.

6. The method of claim 1, wherein said mammal is a cancer patient with a primary brain tumor.

7. The method of claim 1, wherein said mammal is a cancer patient with a metastatic brain tumor.

8. The method of claim 1, wherein said mammal is a patient with an ionizing radiation-treatable lesion.

9. The method of claim 1, wherein said cyclosporin is cyclosporin A or a derivative, metabolite of salt thereof.

10. The method claim 9, wherein said cyclosporin is cyclosporin A.

11. A method for selectively reducing mammal neuron death from ionizing radiation in cyclophilin-rich neurons of central, peripheral, and autonomic nervous systems of a mammal while not reducing damage or death to cyclophilin-poor cells and tissues selected from the group consisting of glia, glia-derived tumor cells, abnormal neuron-derived tumor cells, non-brain tumors, and non-neuron tissue of the body, said method comprising the steps of:
(a) preparing a dosage of cyclosporin A, said dosage being from an effective amount to less than 1 gr/kg of body weight of said mammal; and
(b) administering said dosage to said mammal before, co-incident with, or_after ionizing radiation of said mammal, said dose being administered not later than the same day as the radiation exposure.

12. The method of claim 11, wherein said ionizing radiation comprises a radiation which is selected from the group consisting of alpha radiation, beta radiation, X radiation, gamma radiation, cosmic radiation, fast neutron radiation, proton radiation, and particle beam radiation.

13. The method of claim 11, wherein said ionizing radiation exposure is therapeutic treatment radiation from medical sources, or non-therapeutic radiation from industrial sources, natural sources, man-made sources, or nuclear sources.

14. The method of claim 11, wherein said cyclophilin ligand is administered by parenteral injection, said injection being into or adjacent to, the brain, tumor, or spinal cord, or via cerebrospinal fluid spaces, intraventricular fluid spaces, or intrathecal spaces, or via application into the digestive, respiratory, or genito-urinary systems, or skin, or by a combination of these routes, so that said cyclophilin ligand comes into contact with neurons.

15. The method of claim 11, wherein said mammal is a cancer patient with a primary brain tumor.

16. The method of claim 11, wherein said mammal is a cancer patient with a metastatic brain tumor.

17. The method of claim 11, wherein said mammal is a patient with an ionizing radiation-treatable lesion.
